SEQUENCE LISTING (3 sequences)

SEQ ID NO: 1 - DNA, 18 bp, synthetic construct
  misc_feature 1..18: note = primer_bind
  source 1..18: mol_type = other DNA
agctgtgagtgccctgaa

SEQ ID NO: 2 - DNA, 18 bp, synthetic construct
  misc_feature 1..18: note = primer_bind
  source 1..18: mol_type = other DNA
gtcacagtcggtgccaat

SEQ ID NO: 3 - DNA, 24 bp, synthetic construct
  misc_feature 1..24: note = primer_bind
  source 1..24: mol_type = other DNA
tcctggacgacggtttcatctgca

(19) United States
(12) Patent Application Publication
(10) Pub. No.: US 2024/0425924 A1
(43) Pub. Date: Dec. 26, 2024

(54) KIT FOR EVALUATING THROMBOMODULIN MRNA IN MATERNAL BLOOD FOR THE EARLY PREDICTION OF PRE-ECLAMPSIA

(71) Applicant: PONTIFICIA UNIVERSIDAD JAVERIANA, Bogota, D.C. (CO)

(72) Inventors: Paola Andrea AYALA RAMÍREZ, BOGOTÁ D.C. (CO); Reggie GARCÍA ROBLES, BOGOTÁ D.C. (CO)

(21) Appl. No.: 18/705,551

(22) PCT Filed: Oct. 28, 2022
(86) PCT No.: PCT/IB2022/060421

§ 371 Date: Apr. 27, 2024

(30) Foreign Application Priority Data
Oct. 28, 2021 (CO) NC2021/0014590

(51) Int. Cl. C12Q 1/6883

(52) U.S. Cl. CPC C12Q 1/6883; C12Q 2600/158

(57) ABSTRACT

Disclosed are: nucleic acid molecules of the type messenger RNA (mRNA); kits for detecting thrombomodulin mRNA in maternal blood by means of real-time PCR, which comprise the molecules; and a method for the early screening of pre-eclampsia by detecting thrombomodulin mRNA levels in plasma by means of real-time PCR using the kits.

### TECHNICAL FIELD

The present invention is in the field of medicine, particularly referring to methods for the evaluation and detection of biomarkers that allow for the early determination or prediction of the risk of suffering from preeclampsia. More particularly, it relates to nucleic acids and kits that comprehend them for evaluating thrombomodulin messenger RNA in maternal blood.

### DESCRIPTION OF PRIOR ART

Preeclampsia (PE) is a systemic disorder of unknown etiology that occurs during the second half of pregnancy (after week 20) and is the leading cause of maternal death in Latin American and Caribbean countries (25.7%), being higher in frequency in Colombia (over 30%) (Khan, K. S., et al. WHO analysis of causes of maternal death: a systematic review, 2006). Although the disease begins in an early stage of pregnancy, its clinical manifestations are usually only detected after week 20, and currently, there is no way to identify a pregnant woman who would be at risk of developing it.

The clinical manifestations that allow the diagnosis of PE include an increase in blood pressure, proteinuria, renal insufficiency, altered hepatic function, pulmonary edema, and cerebral or visual symptoms. It has been suggested that these manifestations are secondary to the existence of a state of endothelial dysfunction and hypoperfusion caused by the presence of thrombi in the placental microcirculation and excessive formation of fibrin deposits, which has been proven by the presence of fibrin deposits in the subendothelium of renal glomeruli and spiral arteries, vascular lesions in the placenta, and parenchymal infarctions in women with PE (Ayala-Ramírez, P. et al. Increased tissue factor and thrombomodulin expression and histopathological changes in placentas of pregnancies with preeclampsia., 2016).

Although there are various theories about the histopathological changes observed in the placenta of women with PE, more information is needed on other manifestations that allow a better understanding of the pathophysiology of this disease. The test that is eventually used in current clinical practice for PE screening is the measurement of blood flow in the uterine spiral arteries by Doppler ultrasonography between weeks 11 to 14 of pregnancy. However, the performance of this test is not clear, as it depends on the operator and the equipment used. According to reports, this test presents a sensitivity of 48% and a specificity of 92% for the presence of bilateral notches. Additionally, in a meta-analysis, it is described that the measurement of the pulsatility index of uterine arteries (UtA-PI) in the first trimester has a sensitivity of 47.8% for the identification of early-onset PE, with a false positive rate of 6.1%, and for late-onset PE a detection rate of 21.5%, with a false positive rate of 2.2% (Velauthar, L. First-trimester uterine artery Doppler and adverse pregnancy outcome: A meta-analysis involving 55,974 women, 2014). Because of this, there is a debate on the current utility of this test in clinical practice.

Thrombomodulin (TM) is a transmembrane glycoprotein primarily expressed on endothelial cells towards the vascular lumen and has a natural anticoagulant function as it alters the substrate specificity of thrombin. Additionally, an increased expression of this protein has been found in myofibroblasts of placentas, as well as in the plasma of patients with severe PE, compared to women with normal pregnancies. Similarly, it has been demonstrated that women with PE present an abnormal coagulation state, as indicated by the increased expression of TM, tissue factor pathway inhibitor type I, and pro-coagulant phospholipid in plasma and placenta, compared to women with normal pregnancies.

There are studies related to the detection and early diagnosis of PE, for example, patent US2005/0255114 discloses therapeutic or diagnostic methods based on nucleotide sequences that encode a variety of biomarkers related to PE. In particular, this document describes that the detection of gene expression levels corresponding to such nucleotide sequences, individually or combined, provides a means for the detection of preeclampsia during pregnancy. Additionally, it describes kits for diagnosing patients at high risk of developing preeclampsia.

In the article “Increased Tissue Factor and Thrombomodulin Expression and Histopathological Changes in Placentas of Pregnancies with Preeclampsia” (Ayala-Ramírez P, et al., 2016), the relationship between TM levels and the pathogenesis of preeclampsia was studied, and significant differences were found between the mRNA levels of TM in placentas from women with PE and those from women with normal pregnancies, leading to the conclusion that both pro-coagulant factors and anticoagulants are involved in the pathophysiology of this disease.

Furthermore, in the article “Expression of Tissue Factor and Thrombomodulin in Placentas of Pregnancies Affected by Early-Onset and Late-Onset Preeclampsia” (Ayala-Ramírez P, et al., 2021), the obtained results coincide with the evidence supporting a role of tissue factor (F3) and TM in the pathophysiology of preeclampsia in the placenta, which represents an incentive to carry out more studies to elucidate the role of these molecules in the pathophysiology of preeclampsia and to generate greater knowledge about this disease. For example, it has been observed that the TM present in maternal plasma is derived from the placenta or from endothelial cells subsequent to vascular alterations, meaning that this protein can be a marker of endothelial alterations that show a significant increase in the plasma.

Thus, the progress made so far has allowed the identification of molecules that could be predictors of PE before 20 weeks of gestation, which is relevant since the early identification of at-risk pregnant women would eventually allow prevention strategies, timely diagnosis, follow-up, and proper management, which would positively impact the health of the mother and her offspring.

Although there are various authors who propose the measurement of TM at the protein level as a method to establish women at risk of developing preeclampsia, only one study has reported elevated levels of TM protein in all three trimesters (Prochazka, M. et al. Markers of endothelial activation in preeclampsia, 2015) and it has not been reported in other works that the measurement of TM levels can be useful in the early prediction of preeclampsia. Many protein biomarkers have been associated with preeclampsia, however, to date, no specific protein marker has been found for the prediction of preeclampsia.

This disclosure provides a promising solution through the measurement of TM mRNA levels in maternal blood starting from week 5 and before week 20 of pregnancy, which would allow the identification of pregnant mothers who may later develop PE with a sensitivity of 100% and a specificity of 62%. This type of screening would constitute a valuable tool for the promotion and prevention of maternal health in the first stage of pregnancy and for public health policies and programs aimed at this population. Likewise, the prediction of preeclampsia in early periods of pregnancy, such as the first trimester, would allow effective interventions for the prevention of the disease. The identification of pregnant women at high risk of developing preeclampsia in such periods would allow appropriate follow-up that would eventually improve maternal care and the outcome of pregnancy, which seeks to help reduce the leading cause of maternal death in Latin American and Caribbean countries.

The tests conducted included a pregnant population in general without considering risk factors for preeclampsia, which is why the kit described in this disclosure can be used in populations at low and high risk for preeclampsia.

### BRIEF DESCRIPTION

This disclosure relates to nucleic acid molecules of mRNA type, kits for evaluating of TM mRNA in maternal blood by real-time PCR that comprise these molecules, and a method for the evaluation of preeclampsia through the detection of TM mRNA levels in plasma by real-time PCR with such kits.

### BRIEF DESCRIPTION OF THE FIGURES

FIG. 1. Representation of the hybridization on the thrombomodulin (TM) gene (Genebank ID NM_000361.2).

FIG. 2. Box and whiskers diagram showing the concentration of thrombomodulin (TM) mRNA obtained from plasma, represented on the log10 of MoM. Median of TM mRNA in patients who developed preeclampsia MoM=4.95, minimum and maximum range MoM=1.21-66.29. Median of TM mRNA in patients who did not develop preeclampsia MoM=0.84, minimum and maximum range MoM=0.05-852.10. p-value=0.042.

FIG. 3. Correlation between MoM levels of TM mRNA and the week of sample collection. Spearman's correlation −0.10 (p=0.267)

FIG. 4. ROC curve for thrombomodulin (TM) mRNA with area under the curve 0.745; standard error 0.055; 95% confidence interval from 0.637 to 0.854 and p-value=0.043

### DETAILED DESCRIPTION

For the purposes of interpreting the terms used throughout this document, their usual meaning in the technical field should be considered, unless a specific definition is incorporated, or the context clearly indicates otherwise. Additionally, the terms used in the singular form will also include the plural form.

The present disclosure refers to nucleic acid molecules of the mRNA type designed to hybridize with specific regions of the thrombomodulin (TM) gene (Genebank ID NM_000361.2), which are selected from the group consisting of SEC ID No. 1, SEC ID No. 2, and SEC ID No. 3 as established in Table 1.

TABLE 1
Nucleotide sequences of the designed mRNA molecules

| SEC ID No. | NUCLEOTIDE SEQUENCES |
|---|---|
| 1 | AGCTGTGAGTGCCCTGAA |
| 2 | GTCACAGTCGGTGCCAAT |
| 3 | TCCTGGACGACGGTTTCATCTGCA |

In another aspect of this disclosure, a kit is described for the detection of TM mRNA in maternal blood by real-time PCR (polymerase chain reaction) that includes primers selected from the group consisting of SEC ID No. 1, SEC ID No. 2, and the probe consisting of SEC ID No. 3.

In a particular embodiment, the hydrolysis probe is marked at the 5′ and 3′ ends with different types of radioactive or fluorescent molecules, selected from, but not limited to, nucleotides labeled with ³²P or tritium, digoxigenin, derivatives of fluorescein including fluorescein isothiocyanate (FITC) and 5-(6)-carboxyfluorescein-N-hydroxysuccinimide ester (FAM); derivatives of rhodamine including tetramethyl rhodamine isothiocyanate (TRITC) and Texas Red (sulforhodamine 101 sulfonate); cyanine dyes of wide wavelength range including Cy5.5-Cy7; nanometric crystal particles known as “quantum dots”; 5′-chloro-dimethoxy-fluorescein (JOE), 6-VIC, hexachloro-fluorescein (HEX), 5-TAMRA-5-carboxytetramethylrhodamine, NED, carboxyrhodamine (ROX), carboxy-2,4,7,7-tetra-chlorofluorescein (TET) and fluorescence quenchers or “quenchers” selected from Blackberry (9-[4-nitro-2′,5′-dimethoxy-azobenz-4-yl]-diazo-julolidina-8)-O-hexyl-[2-cianoethyl-(N,N-diisopropyl)]-phosphoramidite), Deep Dark, Dabey, 4-N-methyl-N-(4′-nitro-2′-chloroazobenz-4-ail)-aminobutanamido-1-(2-O-dimethoxytrityloximethyl)-pirrolidin-4-il-O-(2-cianoethyl)-(N,N-diisopropyl)-phosphoramidite (Eclipse), Iowa Black® FQ, lowa Black® RQ, Black Hole Quencher®-1, Black Hole Quencher®-2, succinimidyl ester of QSY 7, acidic dimethylaminonaphthalenesulfonic (Dabsyl), among others.

In a particular embodiment, the probe is labeled with 6-carboxyfluorescein (6-FAM) at the 5′ end and Blackberry Quencher (BBQ) at the 3′ end.

The advantage of using this fluorochrome and this “quencher” is that most real-time PCR thermocyclers have channels to excite and read the emission wavelength of these compounds. Moreover, they are economical and perform well on most equipment.

In an additional aspect, the present disclosure relates to an early screening method for preeclampsia that involves extracting mRNA from maternal plasma and detecting levels of thrombomodulin mRNA using real-time PCR with a kit that includes primers selected from the group consisting of SEC ID No. 1, SEC ID No. 2, and the probe consisting of SEC ID No. 3.

mRNA is extracted from maternal plasma using any method known in the art. The blood sample is taken from pregnant mothers with less than 20 weeks of gestation, more specifically between weeks 8 and 18. Quantitative real-time PCR (QRT-PCR) is carried out with a reaction mix containing a forward primer (5 μM) between 0.125 μM and 0.5 μM final concentration (between 0.25 and 1 microliters in a final volume of 10 microliters); reverse primer (5 μM) between 0.125 μM and 0.5 μM final concentration (between 0.25 and 1 microliters in a final volume of 10 microliters)); probe (2 μM) between 0.062 μM and 0.25 μM final concentration (between 0.125 and 0.25 microliters with a final volume of 10 microliters); activator between 2.5 mM and 3.75 mM final concentration (between 0.5 and 0.75 microliters in a final volume of 10 microliters); master mix 3.7 microliters (in a final volume of 10 microliters) and RNA between 5 and 28 nanograms per microliter (5 microliters in a final volume of 10 microliters).

In a particular embodiment, the reaction mix contains forward primer (5 μM) 0.5 microliters; reverse primer (5 μM) 0.5 microliters; probe (2 μM) 0.25 microliters; activator 0.65 microliters; master mix 3.7 microliters and RNA 5 microliters.

The conditions of the thermocycler for the QRT-PCR are

| | Temperature | Ramp | Time | Number |
|---|---|---|---|---|
| Condition | (° C.) | (° C./second) | (Seconds) | of cycles |
| Reverse Transcriptase | 61-63 | 4.4-4.8 | 60-180 | 1 |
| Initial Denaturation | 94-96 | 4.4-4.8 | 10-30 | 1 |
| Denaturation | 94-96 | 4.4-4.8 | 10-15 | 40-45 |
| Annealing | 58 | 2.2-2.5 | 30-60 | |
| Elongation | 70-72 | 4.4-4.8 | 1-15 | |

In a particular embodiment, the QRT-PCR is performed under the following conditions:

| | Temperature | Ramp | Time | Number |
|---|---|---|---|---|
| Condition | (° C.) | (° C./second) | (Seconds) | of cycles |
| Reverse Transcriptase | 63 | 4.4 | 180 | 1 |
| Initial Denaturation | 95 | 4.4 | 10 | 1 |
| Denaturation | 95 | 4.4 | 10 | 45 |
| Annealing | 58 | 2.2 | 30 | |
| Elongation | 72 | 4.4 | 15 | |

The fragments obtained from the amplification are purified and detected by any method known in the art. In a particular embodiment, the fragments are purified using a commercial kit such as the Wizard® SV Gel and PCR Clean-Up System (Promega, Wisconsin) following the manufacturer's protocol. In another embodiment, the fragments are detected by spectrophotometry, for example, on a Nanodrop 2000® spectrophotometer using nuclease-free water as the blank.

The number of copies per microliter is determined using the formula:

μg/pmol of RNA=(Size in bp of DNA or dsRNA)×(649 g/mol)×(10⁻¹² mol/pmol)×(10⁶ μg/g) Formula 1:

Copies/μl=(ng/μl of RNA/μg/pmol of RNA)×(10⁻¹² mol/pmol)×(10⁻³ g/mg)×(6.022×10²³) Formula 2:

This invention will be presented in detail through the following examples, which are provided solely for illustrative purposes and not with the objective of limiting its scope.

### EXAMPLES

### Example 1. Design of Primers and Probes for the Detection of Thrombomodulin mRNA

The design of the primers and probes was carried out using the Primer 3 program (http://bioinfo.ut.ee/primer3-0.4.0/) and Primer-blast (https://www.ncbi.nlm.nih.gov/tools/primer-blast/) using the sequence with the gene bank access code NM_000361.2.

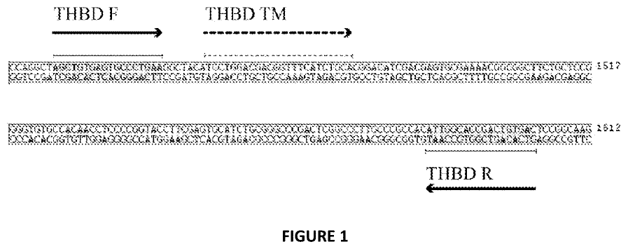

Based on the previous parameters, the sequences listed in Table 2 were obtained, which hybridize with the gene regions of TM ID1 as observed in FIG. 1.

For the execution of the assays, the hydrolysis probe was labeled with 6-carboxyfluorescein (6-FAM) at the 5′ end and BlackBerry® Quencher (BBQ)® (9-[4-nitro-2′,5′-dimethoxy-azo-benz-4′-il]-diazo-julolidina-8)-O-hexyl-[2-cyanoethyl-(N,N-diisopropyl)]-phosphoramidite at the 3′ end.

TABLE 2
Sequences of probes and primers

| Forward | | | Fragment size | Position |
|---|---|---|---|---|
| primer | Reverse primer | Probe | (base pairs) | NM_000361.2 |
| AGCTGTG | GTCACA | BBQ- | 174 | 1430-1603 |
| AGTGCCC | GTCGGT | TCCTGGACGACGGTTTC | | |
| TGAA | GCCAAT | ATCTGCA-FAM | | |

### Example 2. Detection of TM mRNA

### 2.1. Sample Collection and Initial Processing

The primers and the probe from the previously described kit were used. Samples from 106 pregnant women with less than 20 weeks of gestation were collected and followed until their outcome.

The sample was taken using the conventional method, employing a venoject® needle attached to a vacutainer® sleeve; sample collection could also be done using a syringe, taking care to add EDTA anticoagulant immediately after extraction. A total of 4 ml of blood was taken in the tube with anticoagulant (EDTA). The blood sample was processed within 4 hours of collection. It was centrifuged at 3000 rpm for 15 minutes at room temperature, 2 ml of plasma was taken, and transferred into a 15 ml conical tube. Then, 2 ml of guanidine thiocyanate/phenol (QIAzol Lysis Reagent from Qiagen) was added to the sample, it was vortexed and stored at −20° C. until processing (up to a maximum of 1 week).

2.2. mRNA Extraction

For the extraction of mRNA from plasma obtained in 2.1, the miRNeasy serum/plasma kit from Qiagen (Cat. No./ID: 217184) was used. To the 4 ml mixture of plasma/phenol guanidine thiocyanate (QIAzol Lysis Reagent from Qiagen), 400 microliters of chloroform were added and vortexed for 10 seconds. It was centrifuged at 4,000 rpm for 15 minutes at 4° C. The supernatant was transferred to another tube and ethanol 70% of the same amount of the supernatant was added and vortexed. 700 microliters of the obtained mixture were added to the Qiagen column, and the processing was continued according to the manufacturer's instructions.

### 2.3. Quantitative PCR in Real Time (QRT-PCR)

For the amplification of the fragment of interest, quantitative real-time PCR (QRT-PCR) was carried out in a LightCycler® Nano from Roche® using the LightCycler® 480 RNA Master Hydrolysis Probes kit from Roche®, with a reaction mixture of 60 μl as shown in Table 3 and under the conditions of Table 4. Subsequently, the obtained fragment was purified using the Wizard® SV Gel and PCR Clean-Up System (Promega, Wisconsin) following the manufacturer's protocol.

TABLE 3
Reaction mixture for amplification of the fragment of interest for a
final volume of 60 μl:

| Reactive | Volume (μl) |
|---|---|
| Forward Primer (5 micromolar) | 3 |
| Reverse Primer (5 micromolar) | 3 |
| Probe (2 micromolar) | 1.5 |
| Activator | 3.9 |
| Master mix | 22.2 |
| RNA | 12 |
| Water | 14.4 |

TABLE 4
Thermocycler conditions

| | Temperature | Ramp | Time | Number |
|---|---|---|---|---|
| Condition | (° C.) | (° C./second) | (Seconds) | of cycles |
| Reverse Transcriptase | 63 | 4.4 | 180 | 1 |
| Initial Denaturation | 95 | 4.4 | 10 | 1 |
| Denaturation | 95 | 4.4 | 10 | 45 |
| Annealing | 58 | 2.2 | 30 | |
| Elongation | 72 | 4.4 | 15 | |

### 2.4. Preparation of the Standard Curve

For the preparation of the mRNA standard curve, mRNA extracted from placental tissue was used. The extraction was carried out as follows: Approximately 80 mg of placental parenchyma tissue was weighed into a 2-milliliter tube, to which 500 μl of cell lysis solution [0.025M EDTA; 0.069M SDS] and 16 μl of proteinase K [25 mg/ml] were added. The sample was homogenized with an electric homogenizer, and the tissue was incubated at 65° C. for 24 hours in a serological bath. Then, 1 ml of TRIzol (guanidine/phenol) (Invitrogen®) was added and mixed by vortex for 10 seconds. It was incubated at room temperature for 5 minutes. Next, 200 μl of chloroform were added, mixed by vortex for 10 seconds and incubated at room temperature for 3 minutes. It was then centrifuged at 12,000 rpm for 15 minutes, and the aqueous phase was transferred to a 2 ml tube. To this, 500 μl of pure isopropanol were added and mixed, incubated for 10 minutes at room temperature. It was centrifuged at 12,000 rpm for 10 minutes, the supernatant was discarded, and 500 μl of 70% ethanol were added and mixed by inversion. It was centrifuged at 7,500 rpm for 5 minutes, the supernatant was discarded, and 50 μl of RNase-free water were added. It was incubated for 10 minutes at 56° C. in a serological bath and stored at −20° C. until processing (up to 24 hours maximum).

The fragment of interest was amplified according to the QRT-PCR described in section 2.3, and the mRNA was quantified on a Nanodrop 2000® spectrophotometer using RNase-free water as the blank and 2 μl of the sample (between 5 and 28 ng/μl of RNA).

To construct the standard curve, serial dilutions were made in a final volume of 100 μl. The dilutions were 10¹¹, 10⁹, 10⁷, 10⁵, 10³, and 10¹ copies/μl and QRT-PCR was performed in duplicate using the prepared dilutions. The reaction mix for the standard curve was as follows:

TABLE 5
Amplification reaction mix for the mRNA dilutions of the standard curve:

| Reactive | Volume (μl) |
|---|---|
| Forward Primer (5 micromolar) | 0.5 |
| Reverse Primer (5 micromolar) | 0.5 |
| Probe (2 micromolar) | 0.25 |
| Activator | 0.65 |
| Master mix | 3.7 |
| RNA | 1 |
| Water | 3.4 |

With the obtained data, the graph was constructed using the linear equation formula \(y=mx+b \), where \(y \) is the Ct value (cycle threshold) and \(x \) is the concentration in copies/μl. The curve was validated with an \(r \) value above 0.99, a p-value of \(p<0.05 \), and an efficiency above 1.9.

2.5. Determination of mRNA in the Samples

QRT-PCR was carried out from the mRNA sample extracted from plasma as described in section 2.2, together with two samples that correspond to dilutions of the standard curve. A control item was used with an amplified gene \(GAPDH \) and a molecular grade negative control. The reaction mix used was the following, and the thermocycler conditions are described in Table 3.

TABLE 6
Reaction mixture for sample amplification and negative control

| Reactive | Volume (μl) |
|---|---|
| Forward Primer (5 micromolar) | 0.5 |
| Reverse Primer (5 micromolar) | 0.5 |
| Probe (2 micromolar) | 0.25 |
| Activator | 0.65 |
| Master mix | 3.7 |
| RNA or Water | 5 |

The amplification parameters are as follows:
Negative control: no amplification
Internal control: amplifies with a ct less than or equal to 25 cycles
Dilutions: amplifies 10⁵ copies/μl with a ct not greater than 30 cycles, 10⁷ copies/μl with a ct not greater than 25 and/or 10⁹ copies/μl with a ct not greater than 20 cycles.

### Example 3. Results of mRNA Quantification

To establish the number of copies/μl of thrombomodulin (TM) mRNA of each of the samples, the ct values were extrapolated to the standard curve. With these concentrations, the median of all the evaluated samples was found, and for each of the samples, the multiple of the median (MoM) was calculated. The cut-off point to establish the risk of developing preeclampsia is 1.20. That is, women with a MoM equal to or higher than 1.20 are at high risk of developing preeclampsia, or conversely, values below 1.20 MoM imply a low risk of developing preeclampsia.

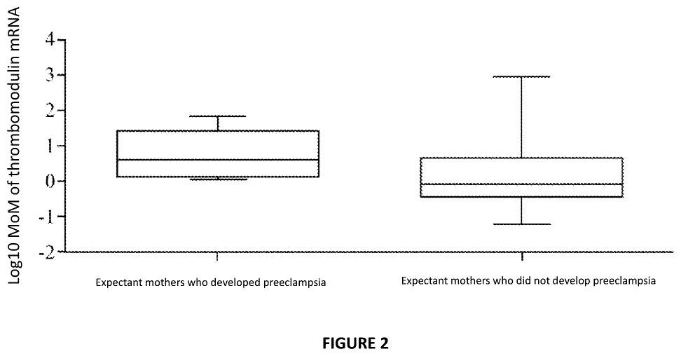

Table 7 and FIG. 2 show the results of the quantification presenting the MoM values. Statistically significant differences were found, comparing the medians with the Mann-Whitney U test, between the groups finding a higher concentration in patients who developed PE with an increase of 5.8 times over the women who did not develop PE.

TABLE 7
MoM of the quantity of mRNA (messenger RNA) of thrombomodulin
obtained from plasma

| Quantification of | Patients who developed | Patients who did not |
|---|---|---|
| thrombomodulin (MoM) | preeclampsia | develop preeclampsia |
| Median | 4.95 | 0.84 |
| Range | 1.21-66.29 | 0.05-852.10 |

| | |
|---|---|
| p-Value | 0.042 |

Additionally, the concentrations of TM mRNA in pregnant women who developed pregnancy-induced hypertension (PIH) were analyzed to determine if the increase in levels was specific to PE or to hypertensive disorders of pregnancy. For this reason, an analysis of TM levels was performed among women who developed PIH (n=10) and those who did not, excluding the 6 women with PE. No statistically significant differences between the groups were evident (p=0.705).

In the same manner, TM levels were analyzed in patients who developed intrauterine growth restriction (IUGR) (n=9); however, no statistically significant differences were found (p=0.606), indicating that the increase in TM mRNA levels before the 20th week could be specific to PE.

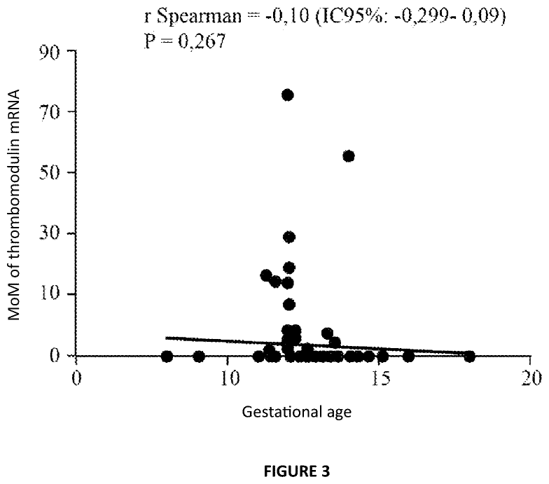

To establish the behavior of the levels of MoM of TM mRNA throughout the weeks of gestation, an analysis of the Spearman correlation coefficient was carried out, as observed in FIG. 3. No correlation was found between the MoM of TM and gestational age.

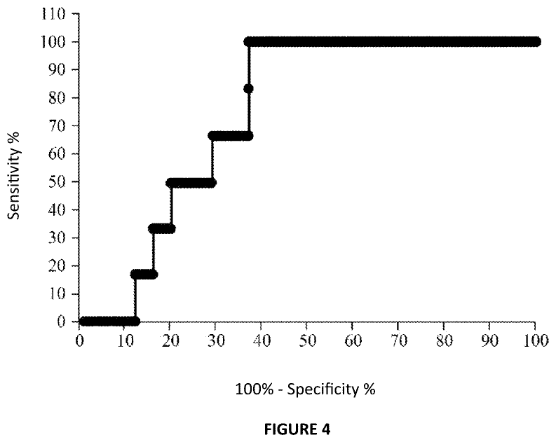

According to the results obtained, a significant increase in TM mRNA levels was observed in patients who developed preeclampsia. Therefore, to assess the degree of specificity and sensitivity at various cut-off points, a ROC (Receiver Operating Characteristic) curve was constructed, as observed in FIG. 4 and Table 8. Additionally, the PPV (positive predictive value), NPV (negative predictive value), and false positives were calculated.

A multiple of the median (MoM) >1.20 was established as the best cutoff point, with a 100% sensitivity and a specificity of 62.6%.

TABLE 8
Contingency table to establish predictive values of the MoM of
thrombomodulin mRNA.

| Expression | | | | | |
|---|---|---|---|---|---|
| of TM | | | | | |
| mRNA in | | | | | |
| plasma | Preeclampsia | | p-Value | | |
| (MoM) | Present (n) | Absent (n) | (Fisher) | OR | 95% CI |
| Result equal | 6 | 37 | 0.003 | 21.6 | 1.18-395.9 |
| to or greater | | | | | |
| than 1.2 | | | | | |
| Result lower | 0 | 62 | | | |
| than 1.2 | | | | | |

Predictive values of TM mRNA:
Positive predictive value (PV)=6/(6+37)*100 =14%
Negative predictive value (NPV)=62/(0+62)*100 =100%
False positives: 37.4%

The use of multiples of the median (MoM) levels for risk assessment is a method more commonly used in biomarker studies because it is easily interpreted by clinicians and allows for control of variability between laboratories. It is also possible to use the reporting of absolute values.

1. A messenger RNA nucleic acid molecule comprising sequences selected from the group consisting of SEQ ID No. 1, SEQ ID No. 2, and SEQ ID No. 3.

2. A kit for evaluating thrombomodulin mRNA in maternal blood for the early prediction of pre-eclampsia by real-time PCR comprising primers selected from the group consisting of SEQ ID No. 1, SEQ ID No. 2, and the probe consisting of SEQ ID No. 3.

3. A method for early screening of preeclampsia comprising:
a—extracting mRNA from maternal plasma;
b—detecting levels of thrombomodulin mRNA by real-time PCR using the kit of claim 2.
